(19) United States
(12) Patent Application Publication
(10) Pub. No.: US 2011/0318755 A1
(43) Pub. Date: Dec. 29, 2011

(54) Universal Testing Platform for Medical Diagnostics

(76) Inventors: Roger N. Piasio, Cumberland Foreside, ME (US); Andrew Wheeler, Saco, ME (US); Christopher Turmel, Portland, ME (US)

(21) Appl. No.: 13/020,485

(22) Filed: Feb. 3, 2011

Related U.S. Application Data
(63) Continuation-in-part of application No. PCT/US2009/052857, filed on Aug. 5, 2009.
(60) Provisional application No. 61/122,610, filed on Dec. 15, 2008.

(51) Int. Cl. G01N 33/53; C12M 1/34; G01N 21/00; G01N 21/75

(52) U.S. Cl. 435/7.9; 422/417; 435/287.2; 436/164

(57) ABSTRACT

The invention relates to analysis of samples (for example, blood, urine, saliva, or swab). Some embodiments include a diagnostic testing platform. The platform may permit the analysis of a plurality of different samples with no or only minor modifications made to the platform. This platform may also reduce the number of steps performed by a user, and offer increased sensitivity and precision. The platform may be integrated with a low-cost instrument and provide an accurate digital analysis of the reaction.

### RELATED APPLICATIONS

This application is a continuation-in-part of PCT/US2009/052857, filed Aug. 5, 2009, which claims priority to U.S. application Ser. No. 12/185,901, filed Aug. 5, 2008 and U.S. Provisional Application No. 61/122,610, filed Dec. 15, 2008. The entire contents of each of these applications are hereby incorporated by reference.

### BACKGROUND

Several testing platforms allow for the analysis of only a single type of sample, and require manual manipulation to activate the test. For example, the Strep A Twist Cassette by Innovacon/ABON has a sample preparation chamber that separates liquid from the test strip until the end user opens the valve by rotating the chamber. The twist cassette platform is specific for a swab sample. The exit valve in the twist cassette is on a circular twisting plastic piece. The configuration of the valve requires manual intervention and the exit hole, located at the bottom of the valve, is small, which may get blocked or clogged by particulates present in the sample. It would be beneficial to have a testing platform which can analyze a variety of samples. It would also be beneficial to have a testing platform that did not require (or at least reduced) manual interaction.

### SUMMARY

In one embodiment, a testing device includes a cassette portion. The cassette portion includes a bottom, a top, and a chamber, for example, a reaction chamber. The reaction chamber has an applicator receiving portion for receiving a sample applicator. A test strip is located between the bottom and the top of the cassette portion. The device includes a passage connecting the reaction chamber and the test strip. The device also a lateral sliding valve laterally moveable from a first position to a second position. In the first position, the laterally sliding valve obstructs the passage. In the second position, the laterally sliding valve allows sample to flow through the passage from the reaction chamber to the test strip. In one embodiment, the linear sliding valve is moved by a stepper motor.

The top of the cassette portion may have a test strip window for viewing a portion of the test strip. The top cassette may also include at least one 2D barcode label. The at least one 2D barcode label may be located on a side of the test strip window.

In one embodiment, the reaction chamber may be cylindrical. In one embodiment, the reaction chamber has a grooved wall. The reaction chamber contains a reagent, for example, a labeled antibody pellet. The reaction chamber may also include a sample mixing apparatus. For example, the sample mixing apparatus includes a magnet and a magnetic stirring motor designed to mix the labeled antibody pellet and the sample.

In one embodiment, the test strip includes a sample receiving pad, a nitrocellulose membrane, and an absorbent pad. The test strip may also include a bridge pad.

In one embodiment, the applicator receiving portion of the reaction chamber is a swab cone for receiving a swab applicator. In another embodiment, the applicator receiving portion of the reaction chamber is a saliva net for receiving a saliva collector. In another embodiment, the applicator receiving portion is a liquid filter. In another embodiment, the applicator receiving portion is a buffer pad. In another embodiment, the applicator receiving portion is at least one blood separation pad.

In one embodiment, a sample is applied to the applicator receiving portion of the chamber of the test device. The sample is processed in the applicator receiving portion to prepare a processed sample. The processed sample and a reagent are mixed in the reaction chamber to prepare a mixed sample. In one embodiment, the processed sample and the reagent are mixed using sample mixing apparatus, for example, a magnet located in the reaction chamber and a magnetic stirring motor. The mixed sample from the reaction chamber to a test strip of the test device by laterally moving a lateral sliding valve in the test device from a first position to a second position. In one embodiment, the lateral sliding valve is moved from the first position to the second position by an instrument.

In one embodiment, the sample is processed by extracting saliva from a swab or saliva collector. In an alternative embodiment, the sample is processed by filtering and buffering a urine sample. In another embodiment, the sample is processed by separating red blood cells from a remainder of the sample.

The test strip may be analyzed using an instrument. In one embodiment, the test strip is analyzed to determine a positive result or negative result. In another embodiment, the test strip is analyzed to detect the concentration level of an analyte in the sample. In another embodiment, the test device is analyzed to read a bar code.

### BRIEF DESCRIPTION OF THE DRAWINGS

FIG. 1 is a top view of the bottom cassette portion of the test cassette.

FIG. 2 is a top view of the top cassette.

FIG. 3 is a top view of the top cassette assembly.

FIG. 4 is a bottom view of an alternative bottom cassette portion.

FIG. 5 is a top view of a cassette with barcodes.

FIG. 6 is a 3D view of a swab cone.

FIG. 7 is a top view of a saliva collector net and filters holder.

FIG. 8 is a top view of the cassette assembly with a labeled antibody pellet and magnetic stirrer.

FIG. 9 shows the linear valve of the cassette coupled to the stepper motor of the instrument.

FIG. 10a shows a first position of a linear sliding valve during sample incubation.

FIG. 10b shows a second position of a linear sliding valve during sample run.

FIG. 10c shows a third position of a linear sliding valve with conjugates running downstream.

FIG. 11a is a side view of the device of FIG. 10a.

FIG. 11b is a side view of the device of FIG. 10b.

FIG. 11c is a side view of the device of FIG. 10c at the start of the conjugate reverse run step.

FIG. 11d is a side view of the device of FIG. 10c during the conjugate reverse run step.

FIG. 11e is a side view of the device of FIG. 10c showing an antigen capture read line and a control read line.

FIG. 12a shows a first position of a two-stage sliding linear valve during sample incubation.

FIG. 12b shows a second position of a two-stage sliding linear valve during sample run.

FIG. 12c shows a third position of a two-stage sliding linear valve with chase buffer.

FIG. 13a shows a first position of a linear sliding valve during reverse flow conjugate application.

FIG. 13b shows a second position of a linear sliding valve during reverse flow conjugate application—sample run upstream.

FIG. 13c shows a third position of a linear sliding valve during reverse flow conjugate application—conjugate run downstream.

FIG. 14a shows a first position of a linear sliding valve during reverse flow of biotin conjugate and Au conjugate after sample application.

FIG. 14b shows a first position of a linear sliding valve during sample run.

FIG. 14c shows a second position of a linear sliding valve during simultaneous release of biotin conjugate and neutravidin conjugate chase buffers.

FIG. 15a shows a first position of a linear sliding valve during reverse flow conjugate application during sample incubation.

FIG. 15b shows a second position of a linear sliding valve during reverse flow conjugate application with sample run upstream.

FIG. 15c shows a first position of a linear sliding valve during reverse flow conjugate application with both conjugates run downstream.

FIG. 15d is a side view of FIG. 15c.

FIG. 16a is a side view of a blood sample assay cassette with sample/red blood cell separation on the test strip.

FIG. 16b is a side view of a blood sample assay cassette with sample/red blood cell separation in the reaction chamber.

FIG. 16c is a side view of a blood sample assay cassette with buffer storage and sample/red blood cell separation in the reaction chamber.

FIG. 16d is a side view of a blood sample assay cassette with buffer storage in the reaction chamber.

### DETAILED DESCRIPTION

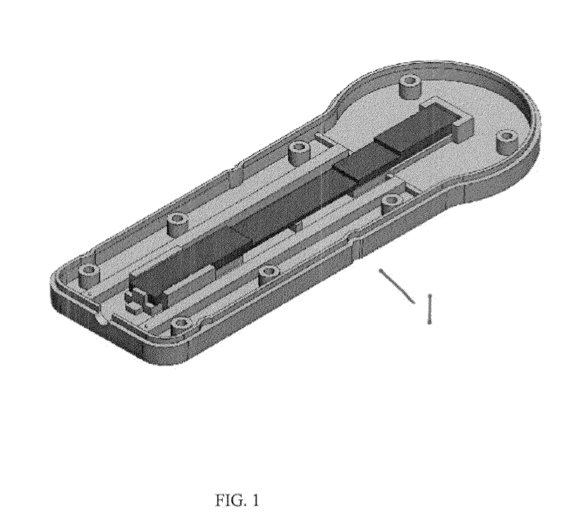

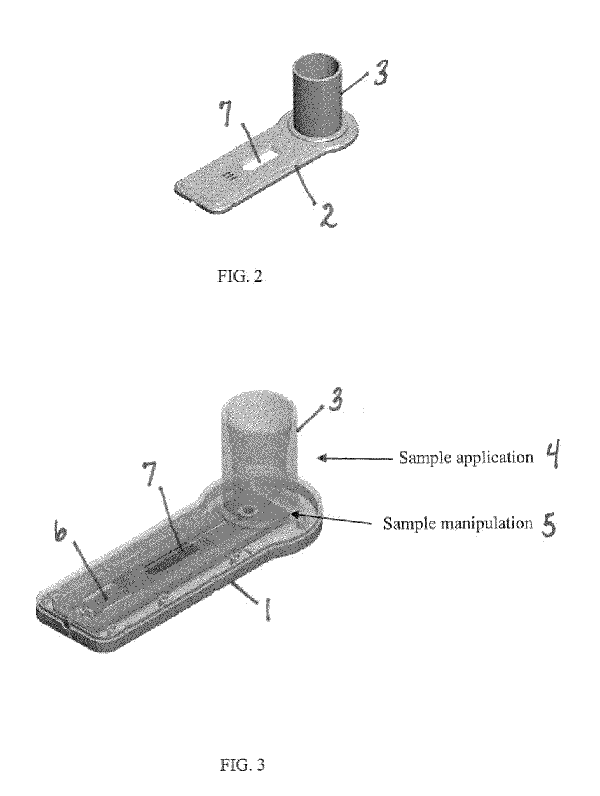

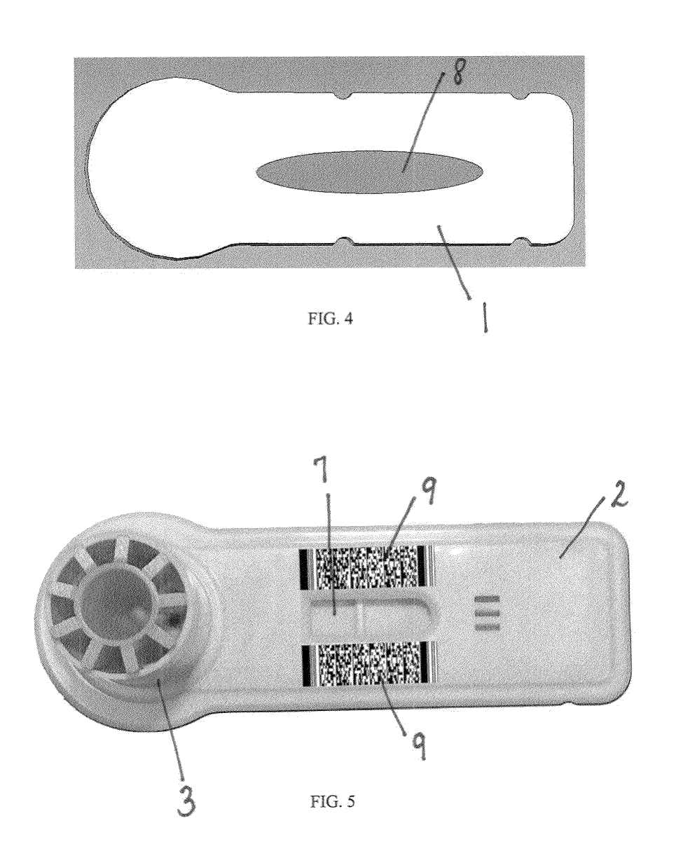

In one embodiment, the instrument-based diagnostic test comprises a cassette, an instrument for reading the cassette, and an applicator for applying sample to the cassette. Referring to FIGS. 1-3, the cassette is composed of a bottom cassette portion 1, a top cassette portion 2, and a reaction chamber 3. In one embodiment, the reaction chamber 3 is cylindrical or barrel-shaped. The reaction chamber 3 has an applicator receiving portion 4 for receiving a sample applicator.

The bottom cassette portion 1 holds a test strip 6. The test strip 6 is located between the bottom cassette portion 1 and the top cassette portion 2. The test strip lies flat along the bottom cassette 1. The top cassette portion 2 has a viewing window 7 for viewing a portion of the test strip 6. The test strip 6 includes a sample receiving pad, a nitrocellulose membrane, and an absorbent pad. In one embodiment, the test strip 6 also includes a bridge pad. In one embodiment, the test strip 6 contains antibodies dried onto the nitrocellulose membrane at specific locations. The test strip 6 is sized to fit within the bottom chamber 1, for example, about 5 mm in width and about 60 mm in length. The cassette can be modified to accept test strips of various lengths and widths.

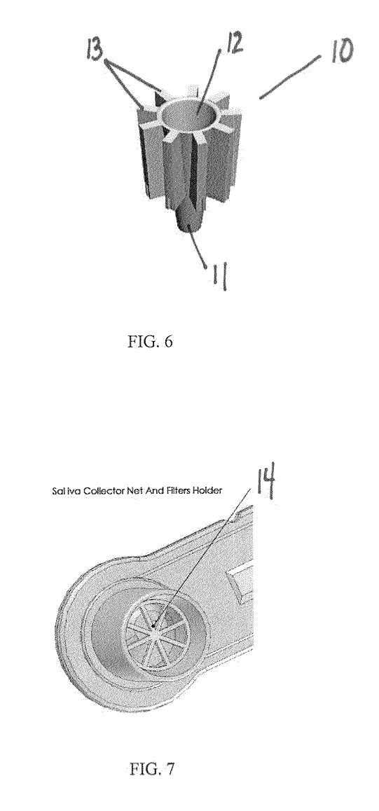

In one embodiment, shown in FIG. 4, the bottom cassette 1 has an oval cutout 8 so that light can be illuminated from the bottom and detected from above. In this embodiment, the test strip is typically made out of a clear backing material. This embodiment allows light transmission and/or light reflectance, to be measured.

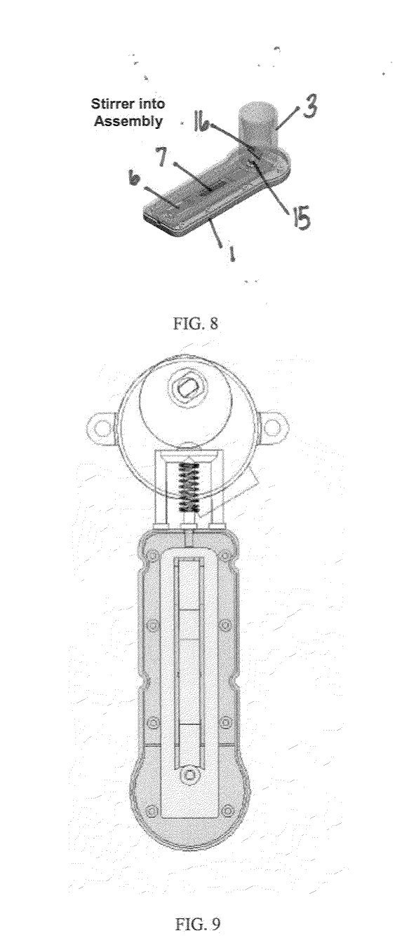

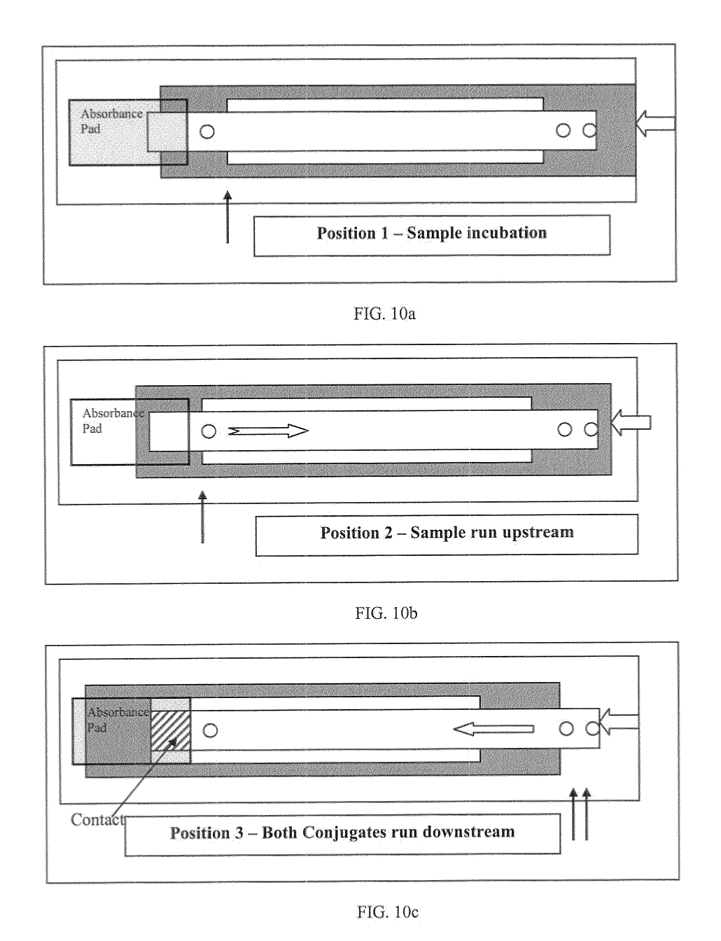

As shown in FIG. 5, the top cassette 2 may also include 2D barcode labels 9, for example, on either side of the test strip window 7. These barcodes 9 are readable by the instrument. In one embodiment, the barcode 9 is the same general focal length as the test strip to minimize errors.

The reaction chamber has a wide grooved wall for accepting a variety of sample applicators, which are described below. Each of the applicator receiving portions described below is designed to pre-process the sample prior to the sample entering the sample manipulation zone, for example, by extracting saliva from a swab or saliva collector, filtering and buffering a urine sample, or separating red blood cells from the remainder of the blood sample. The applicator receiving portion is also designed to keep sample added to the reaction chamber away from a sample manipulation zone.

In one embodiment, the applicator receiving portion of the reaction chamber is a swab cone 10 for receiving a swab applicator. A user inserts a swab into the reaction chamber and then adds buffer to the chamber. With a dry swab inserted, about 300 uL of liquid can be added to the cone without spill. The swab cone 10 blocks the flow of liquid until the swab is removed. This allows for better transport of material off of the swab because the swab is submerged in buffer. The swab blocks the bottom hole 11 of the cone 10 until it is removed so that the swab can be mixed and swirled against the outer wall of the cone. The swab cone's smooth inner surface 12 allows liquid to flow easily down its walls. Having the sample application zone separate from the bottom of the chamber allows for other activities to take place in the bottom of the chamber, thus creating two distinct zones, a sample application zone and a sample manipulation zone. The diameter and height of the cone allows both foam tipped and polyester swabs to be inserted. The swab cone 10 has outside fins 13, which serve to lock it into place against the grooved wall of the top cassette. The fins 13 also allow for air to travel up and down the reaction chamber to stop air locks from occurring, and keep a magnet and a reaction pellet located at the bottom of the reaction chamber in place.

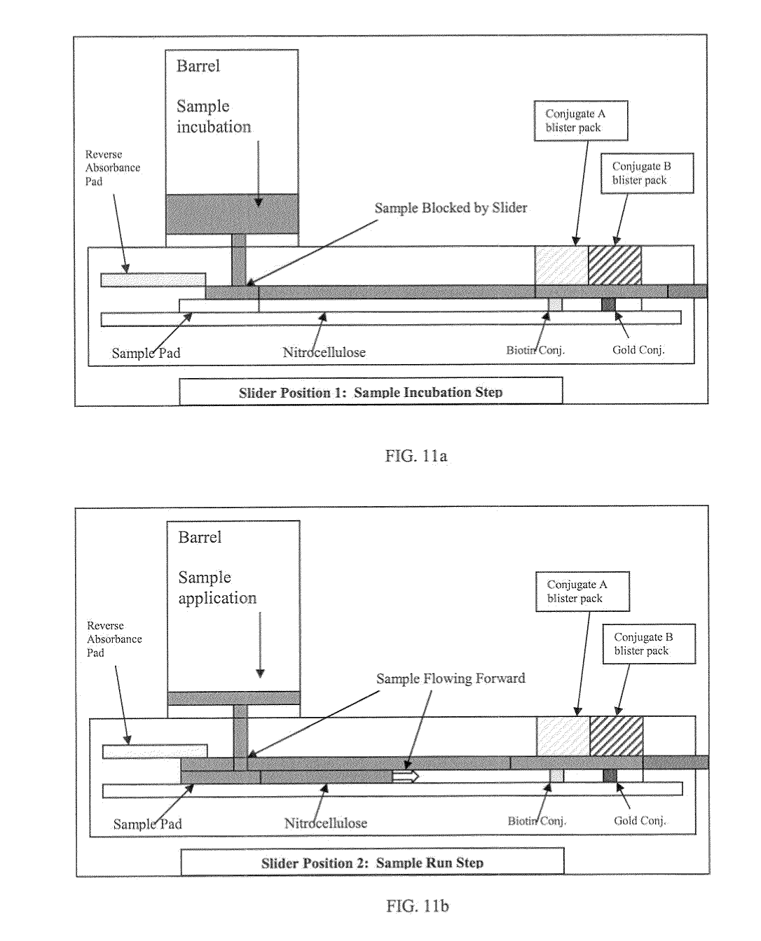

In another embodiment, the applicator receiving portion is a saliva net 14 as shown in FIG. 7. The saliva net applicator receiving portion 14 allows an external saliva foam collector to be squeezed against the plastic saliva net, allowing saliva to expelled. A filter pad can also be inserted on top of or below the saliva net. The saliva net 14 requires only a small amount of saliva to be collected on the saliva foam collector, because all of the liquid from the saliva foam collector is expelled directly into the sample manipulation zone. This can be a major advantage for tests conducted on people with dry mouth.

In another embodiment, the applicator receiving portion is a liquid filter or buffer pad. A disc is placed into the reaction chamber and locked into place with the grooved wall. Preferably, a 9/16 inch disc of Porex material number 1342 ( 1/16″ thick) is used. The disc is dipped into a detergent buffer and dried to remove moisture. This applicator receiving portion allows for urine to be added directly to the platform without the need to use a filtering swab, or to dilute with buffer.

In another embodiment, the applicator receiving portion is a blood separation and collection receiving portion. Separation of a blood sample can occur in the barrel of the reaction chamber of the device, or on the test strip. For separating the blood sample in the reaction chamber, there may be a sample application pad made of a material that separates red blood cells from the remainder of the sample is located at the sample application point inside the chamber. Alternatively, there may be a series of separator pads that have differing properties that will transfer the sample without the red blood cells from the sample collection device (for example, a capillary tube) to the labeled antibody pellet in the reaction chamber. For separating the blood sample on the strip, the sample pad under the reaction chamber is made of a blood separation material or a series of blood separators of differing properties that transfer the sample from the reaction chamber to the test strip while retarding or immobilizing the red blood cells. Examples of separation pad materials that may be used include, for example, VF1, VF2, MF1, and LF1 materials (GE Whatman) or Cytosep (Pall) material. In one embodiment, the sample pads are a series of red blood cell separation pads that immobilize the red blood cells in a flowing sample and keep them from staining the nitrocellulose. In another embodiment, the sample pad(s) are a single pad that separates the blood sample, or two or more pads in a series to accomplish red blood cell immobilization. Antibody against red blood cells can be added to one or more of the pads to assist in the immobilization of the red blood cells.

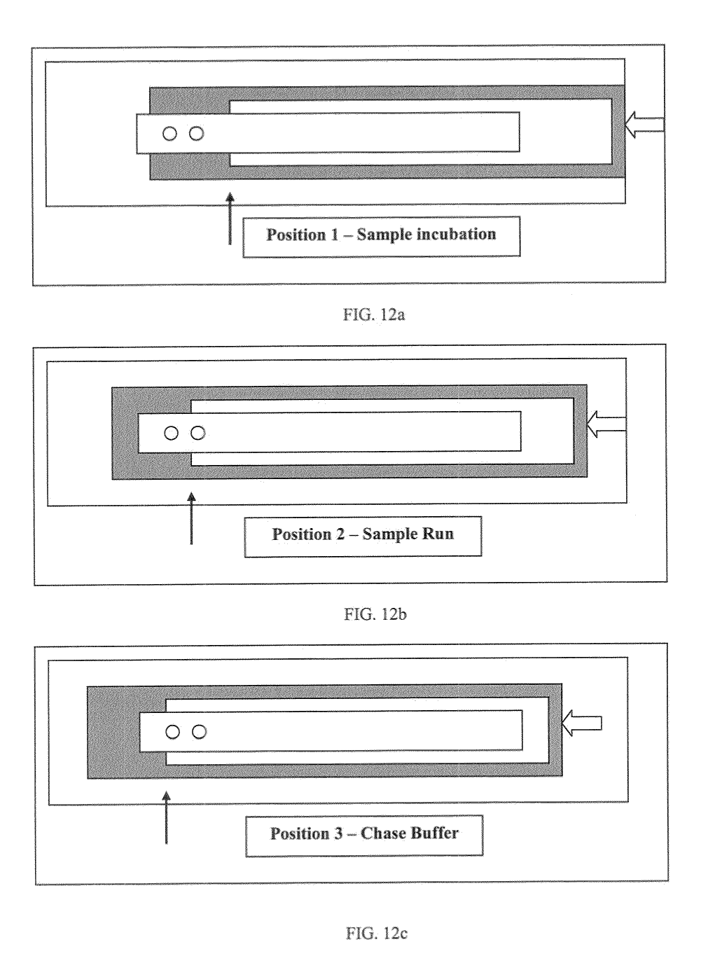

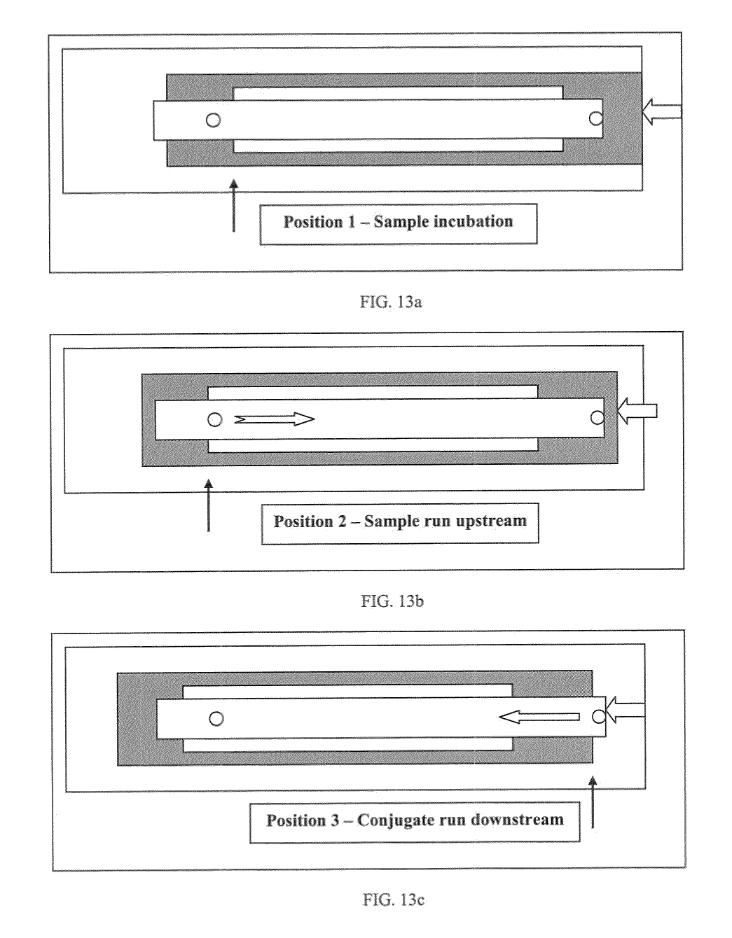

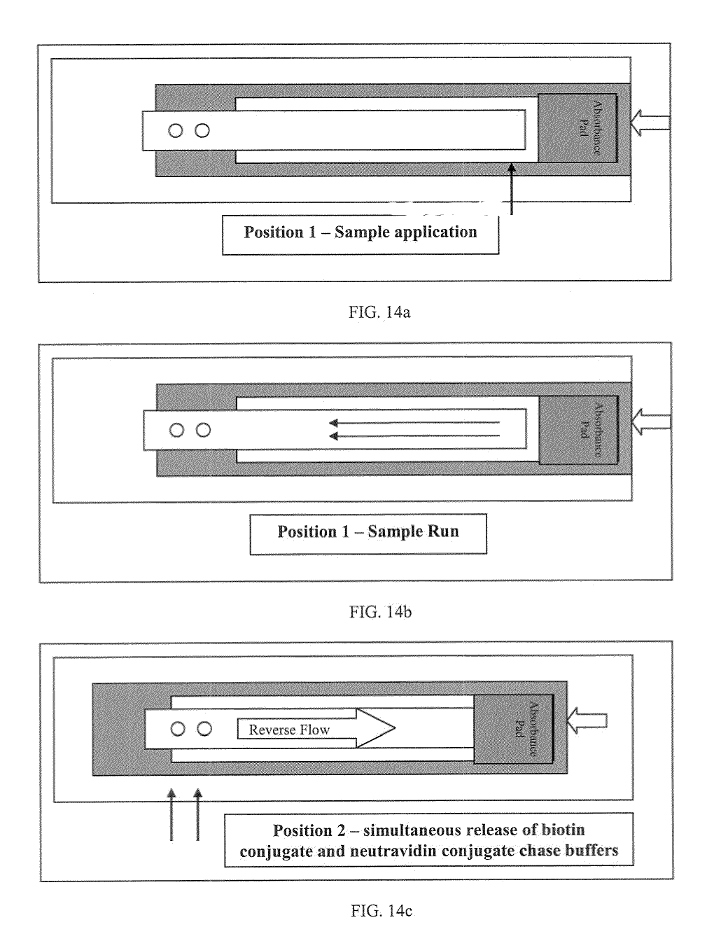

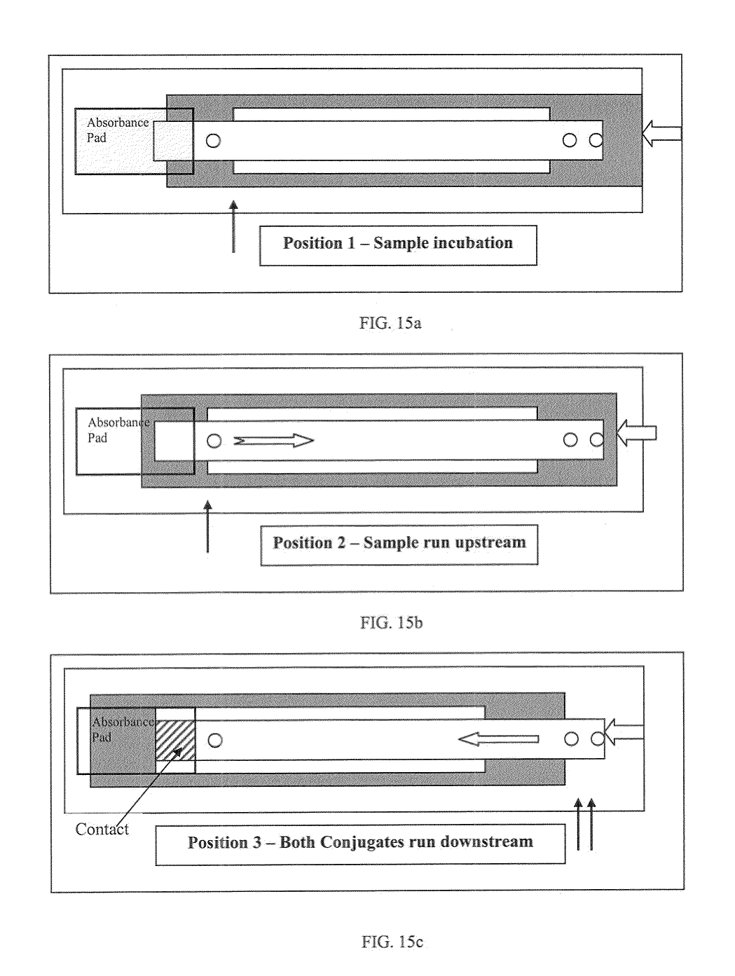

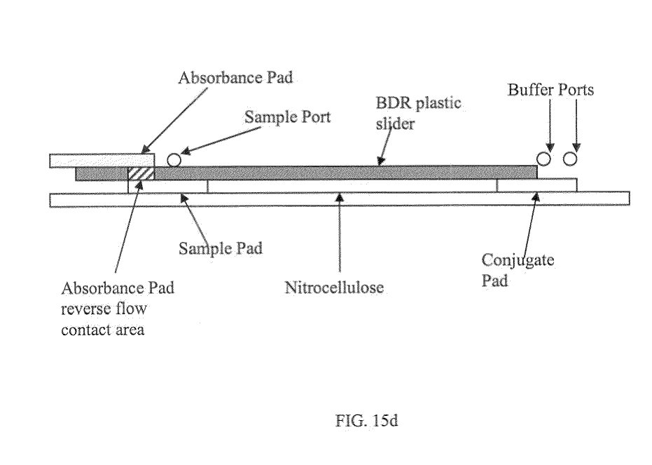

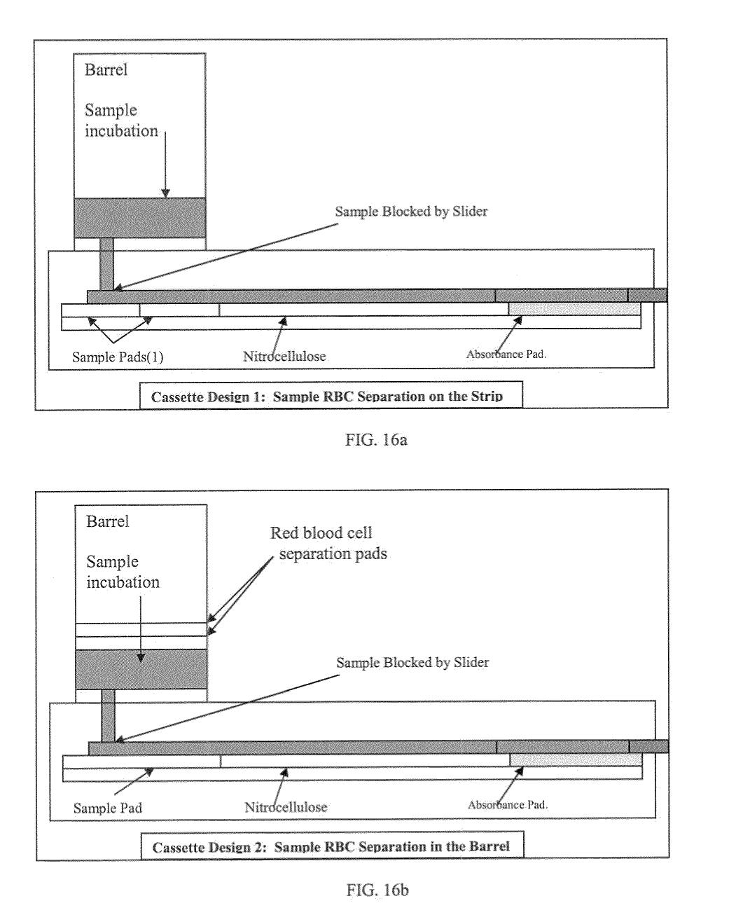

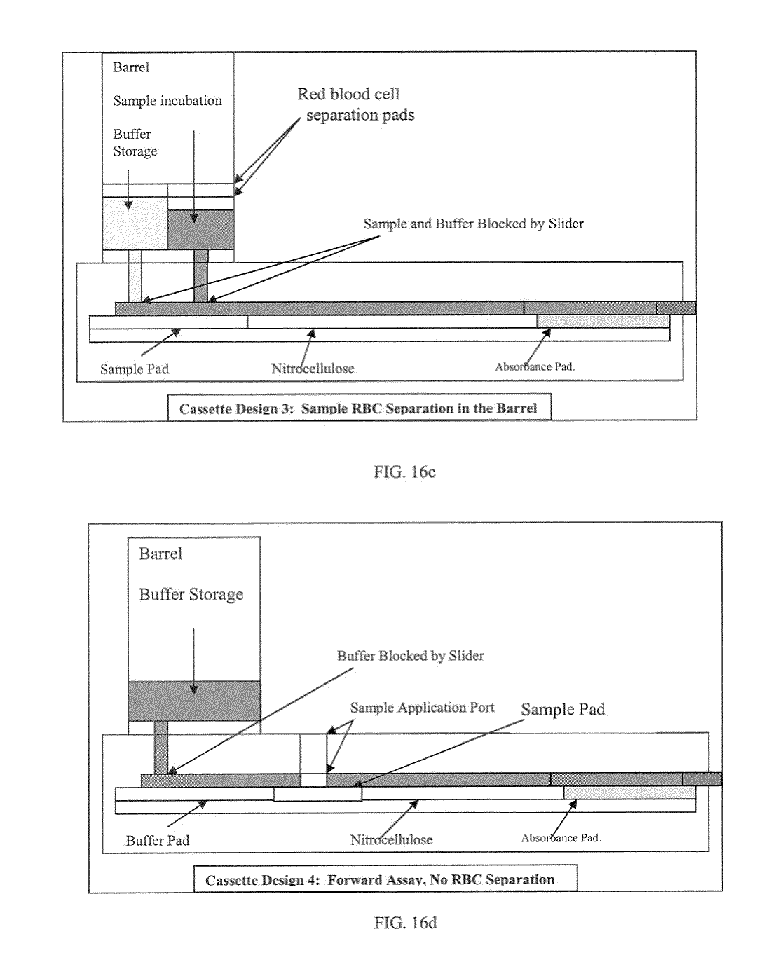

The reaction chamber contains a valve which operates via a linear sliding plastic piece. The valve hole is tapered, and the bottom cassette is adapted to push the sample pad of the test strip closer to the exit hole of the valve. The linear sliding valve allows the step of opening the valve to be automated. This not only reduces the number of steps to be performed by a user, but also reduces technician error due to incorrect timing or incorrect opening of the valve. The valve hole located on the linear valve does not line up with the hole on the top of the cassette until the stepper motor of the instrument pushes the linear sliding plastic piece into place. The top cassette holds the linear sliding valve in place. The space surrounding the linear sliding valve is sealed with an O-ring. The linear sliding valve allows for two separate zones: one for sample application, and one for sample manipulation. FIGS. 10-16 show alternative embodiments of the test cassette. In one embodiment, the linear sliding valve has three stages: closed, open, and closed. This allows the amount of liquid to be controlled based on time. For example, the stepper motor of the instrument would push the valve to the open position and then, based on empirical time testing, would push the valve to the second closed position when the required amount of time had passed.

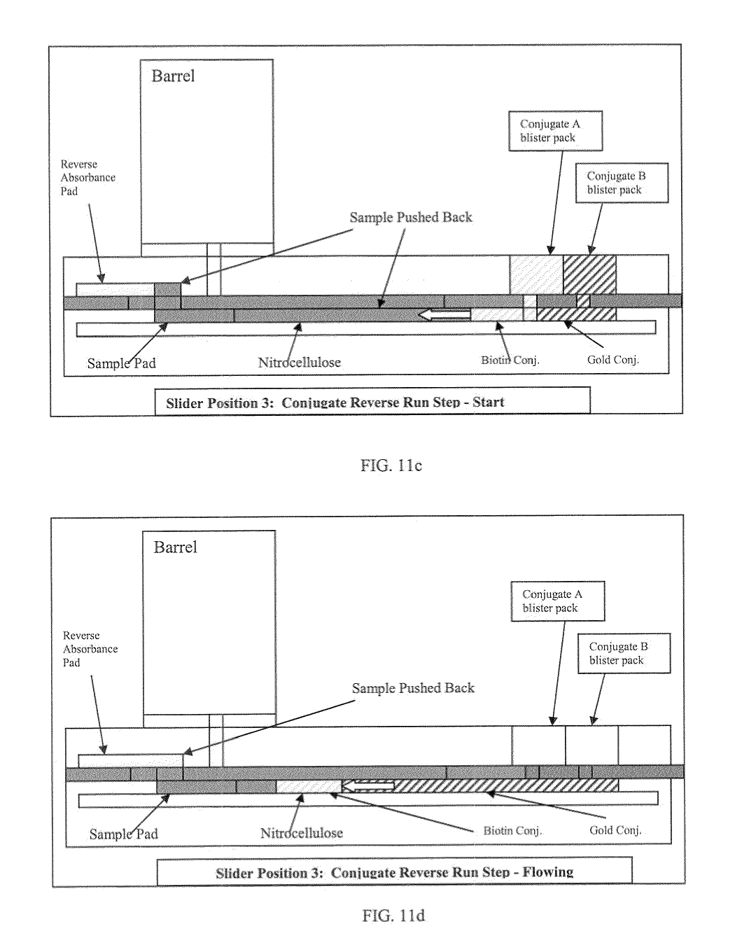

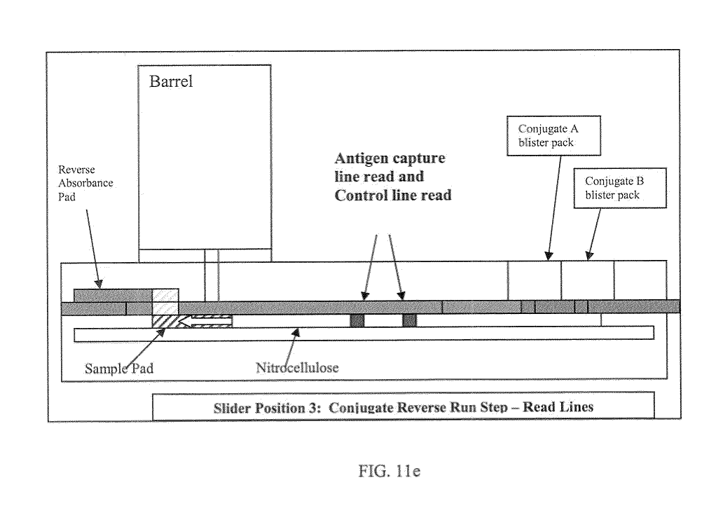

Referring to FIG. 8, the reaction chamber contains a labeled antibody pellet 15 located in the bottom of the reaction chamber 3. The reagent pellet 15 contains components to treat the sample in the sample manipulation zone and create a detectable reaction. The components could include antibody conjugated to a gold colloid, an extraction enzyme, protecting proteins and buffering reagents. In one embodiment, the labeled antibody pellet is a gold bead containing antibodies conjugated to gold and a PlyC enzyme. Using a reagent pellet 15 instead of absorbing labeled antibodies onto the test strip allows for one hundred percent of the sample to mix with one hundred percent of the antibody conjugate. This decreases imprecision due to reconstitution, and kinetics. The labeled antibody pellet 15 increases sensitivity and precision of the device, because it allows the device to detect low levels of analyte and decreases the likelihood of variation. No user steps, such as transferring liquid, are needed to use the device. The labeled antibody pellet 15 may be inserted into the cassette barrel 3 at the time of manufacture. In one embodiment, an exothermic chemical reaction could be included with the reagent pellet 15 to produce a warm liquid prior to chromatography. In this embodiment, the linear sliding valve would help regulate the temperature by opening the valve.

A sample mixing apparatus is also located on the bottom of the reaction chamber 3, as shown in FIG. 8. The function of the sample mixing apparatus is controlled by the instrument. Mixing is accomplished by a small disposable magnet 16 and a magnetic stirring motor. The magnet 16 may be inserted into the cassette barrel at the time of manufacture. Any shape of magnet can be used. In one embodiment, the magnet 16 is a trapezoid. The magnet allows for the mixing of samples with buffer, extraction reagents, and or labeled antibody and eliminates uniformity problems with viscous samples. The speed, duration and action of the stir is fully controllable by software on the instrument. This is useful for mixing viscous samples with the reagent pellet and for increasing kinetics of antigen-antibody interactions. In addition, complete sample preparation is assured prior to chromatography.

Once the sample has been mixed and incubated with the reagent pellet for the proper amount of time, the linear sliding valve is pushed open by the stepper motor of the instrument. When the valve is opened, the liquid solution travels through the valve hole and onto the sample receiving pad of the test strip. Chromatography begins and the reaction is measured. In one embodiment, a chase buffer (or push buffer) is added to the reaction chamber after the sample is loaded or after the incubation time is complete. In another embodiment, the chase buffer or push buffer is included in the reaction chamber and is applied to the test strip with the sample or after the sample using an offset valve port on the slider and a second slide of the slider when programmed. In one embodiment, conjugate buffers are contained in blister packs.

The instrument employs the use of an intelligent CMOS camera with several algorithms and decision trees to detect the signal. The instrument analyzes the test strip every 10-15 seconds until the criteria is met, for example, a positive or negative result is determined, or a certain analyte concentration level is detected. Software in the instrument also allows the location of the detection lines to be altered without changing the design of the system. The instrument can connect via Bluetooth to the software for automatic download of data.

### EXAMPLES

### Example 1

### Instrument-Based Group a Streptococcus Test

The instrument-based Strep A test uses a rapid immunochromatographic cassette and an instrument to detect Group A streptococcus from throat swabs. Traditional Strep A tests utilize micronitrous acid extraction to liberate the group A antigen. This method is both time consuming (1-2 minutes) and is not complete (Kholy et al., “Simplified Extraction Procedure for Serological Grouping of Beta-Hemolytic Streptococci,” Applied Microbiology, November 1974, Vol. 28, No. 5, p. 836-839.). In contrast, the present device utilizes a phage-associated lysin PlyC to extract the group A antigen. The PlyC has been shown to provide complete hydrolysis within a matter of seconds. The present test utilizes a foam-tipped swab instead of the woven polyester swabs used in traditional tests. The benefits of this change include an increase in sensitivity, less user discomfort, and no requirement for transport media. The present test also utilizes a low-cost intelligent camera-based instrument that performs the steps of mixing of sample, automatic incubation of the sample, and kinetic-based analysis of the reaction. The instrument reduces the number of steps to be performed, eliminates ambiguity with low-level signals, provides quicker results, and eliminates transcription errors by transmitting results directly to patient records.

The instrument-based Strep A test also employs antibodies that are more sensitive and more specific than most tests currently on the market. A sheep antibody laid down on the nitrocellulose in a wide line increases the capture line efficiency at limit of detection and is pre-scrubbed against cross-reacting bacteria. The label used is a gold colloid and the sample is mixed with the gold prior to chromatography. The antibody used on the gold particle (NHD 18A) has also shown to be the most sensitive and specific anti-Strep A antibody.

### Identification of Reagents:

Sheep anti-Strep A: BAA, this antibody was produced by ADAPT using an ABBOTT immunogen. Since this study, the ABBOTT immunogen has been replaced with one made by Binax. This antibody has been scrubbed against three different strains of Neisseria to help eliminate cross reactivity prior to purification.

Rabbit anti-Strep A (18A): The current antibody is provided by New Horizons Diagnostics.

Lysin: The recombinant lysin is currently provided by New Horizons Diagnostics. The protein is expressed in E. coli cells and is purified on a hydroxyl apatite column.

Swab: The foam swab from Puritan has been documented to increase sensitivity. If it is not used, then the test can expect to lose about a ½ log in analytical sensitivity. It should also be noted that all of the above testing used this swab.

### Feasilibity:

Results that support the feasibility of the instrument-based Strep A test are shown below. Feasibility is shown by providing the following information:
Identification of critical reagents
Demonstration of reasonable analytical sensitivity
Demonstration of reasonable specificity
Demonstration of detection of target analyte in intended matrix (Clinical samples)
Comparison of low-cost instrument to higher cost gold standard instrument

The purpose of this example is to present data establishing the feasibility of the instrument-based Strep A test. Feasibility has been achieved by identifying specific antibodies and reagents for development and by demonstrating acceptable analytical sensitivity and specificity of the target analyte in the intended matrix.

### Materials:

Instrument-Based Strep A Cassettes:

RTG-A: Instrument

RTK-Top Cassette

RTK-O ring

RTK-Linear valve

RTK-Strep A test strip
G&L Laminated Backing (GL-51954)
Nitrocellulose Sartorius CN95
   Cat#1UN95ER050025WS
   Lot#0308 19910 0703883
ADAPT Sheep anti Strep A (BAA#021)
   1 mg/mL with 0.5% Trehalose 4 uL/cm
Chicken IgY (1 mg/mL luL/cm)
Ahlstrom 1281 12.5 mm wide sample pad
Porex 4588 12 mm wide bridge pad
Ahlstrom 901 18 mm wide absorbent pad

RTK-Bottom Cassette

RTK-Magnet

RTK-Step A gold pellet (Processed by Biosite)
Rabbit anti Strep A conjugate Lot 030948RD
(Acid Clone 180D30, OD 9.375/test)
Donkey anti Chicken conjugate (OD 30, OD 1.4/test)
   1 mg/mL PlyC (12.5 ug/test)
Next Gen bead buffer
   9.5 mg/mL Tetra Borate
   50 mg/mL fraction five bsa
   300 mg/mL Sucrose
   12.5 mg/mL De-aggregated Rabbit IgG
   1 mg/mL Azide
   pH 7.2

RTK-Cone

Elution Buffer—R&D Formulation
0.025M Sodium Citrate
0.25M Tris Base
0.025M EGTA
0.25% SDS
0.25% Nonidet P40
0.05% Sodium Azide
pH 7.2

Swabs: Foam tipped applicator (Puritan medical REF 25-1506 1PF solid)

Strep A dilution buffer: 1×PBS+0.05% Nonidet P40

Instrument: RTG-A serial #6, Strep A program

| | | TestType ID | 0 |
|---|---|---|---|
| Main Board ID | 6 | Test Name String | Strep-A |
| DeviceID | 6 | Thresholds 1-4 | 1200 |
| ScreenType | 0 | Control Threshold | 5000 |
| ScreenVoltage | 0 | Name of Left Line1 | Strep-A |
| BarcodeCheck | 0 | NI String | NO |
| | | | CONTROL |
| Lang | 0 | Minus/Minus String | NEGATIVE |
| DefaultTest | 0 | Plus/Minus String | POSITIVE |
| Message | RTR-Strep | Minus/Plus String | POSITIVE |
| | A | | |
| Home Steps | 0 | Plus/Plus String | POSITIVE |
| Open Steps | 1050 | Line1 Left/Right | 282,603 |
| Return Steps | 0 | Control Left/Right | 640,900 |
| Remove Well | 0 | LED 1-3 | 0, 32, 32 |
| VibratorCLK | 50 | Test Window X/Y | 212,902 |
| VibratorPwr | 1 | Shutter Width | 2,500 |
| ReaderType | 1 | Gain | 16 |
| ShutterDelay | 0 | Stride | 0 |
| Downkey | 4 | Energy | 40,000 |
| SensorType | 2 | Test Image Width/Height | 1,024,210 |
| Checksum | 12 | Division Factor | 40,000 |
| | | Line 1 Type N = 0 W = 1 | 1 |
| | | UpperBound/LowerBound | 30, 30 |
| | | Incubation time (s) | 2 (×10) |
| | | Wait time | 58 (×10) |
| | | Vibrator | 9 |

### Method:

In this example, the instrument-based Strep A test comprises the cassette, the instrument, the swab, and the elution solution. The cassette comprises a test strip, a magnet, a gold bead and a swab cone. The test strip is composed of a nitrocellulose membrane, a sample pad, a bridge pad and an absorbent pad. An anti-Group A Streptococcus antibody provided by ADAPT BAA, and a control antibody, chicken IgY, are absorbed onto the membrane at specific locations. The test strip is cut at 5 mm in width and is 59.5 mm in length. The gold bead comprises rabbit anti-Strep A conjugated to gold, donkey anti-chicken conjugated to gold and the PlyC enzyme. The gold bead and magnet are inserted into the cassette barrel at the time of manufacture. The swab cone is inserted over the gold bead and magnet to lock them into place. The swab cone offers an elution zone that blocks the flow of liquid until the swab is removed. The instrument accepts the cassette, mixes the sample with the gold bead and automatically pushes the linear valve to initiate flow. The instrument automatically analyzes the reaction every 10-15 sec and reports the result based on threshold criteria. The instrument connects via Bluetooth to PC software for automatic download of data.

For analytical testing, 10 uL of Strep A solution was added to the swab. The user will insert the swab into the patient's mouth and will sample the tonsils area. Then the user will insert the swab into the swab cone on the cassette. Elution solution is then added to the cone and the swab is mixed. Elution solution is currently added by disposable transfer pipette ˜200 uL.

### Assay Protocol:

The cassette is removed from its packaging The cassette is placed into the instrument, and the instrument detects the information required to run the test. The swab is placed into the cone on the cassette. The elution solution is added to the cone. The swab is swirled several times (approximately 5 times) and removed. The swab is re-inserted into the cone and swirled around the rim of the cone to remove any excess liquid from the swab. It is then removed for the final time and discarded. The test button on the instrument is pushed and the assay is started. The result is reported via the instrument view screen (or via Bluetooth to a PC) when a signal is detected or the total end point criteria (control line intensity or time) has been reached.

### Example 2

### Analytical Sensitivity Study

Group A Streptococcus (ATCC #19615) was evaluated during this study. Stock solutions diluted from the same frozen aliquot by two different individuals were used. Dilutions were prepared in Strep A dilution buffer. The test was stopped and identified as positive when the signal went above 1000-1200 units. Therefore, time (instead of signal) generates the dose response curve. The assay was stopped (if there was no signal above 1000-1200 units) at 6 minutes total time (340 seconds+20 seconds incubation time). There was determined to be variation between the two stock solutions. This is most likely due to vial to vial variation. One of the 1×10³org/test runs stopped at 326 seconds instead of ˜360 seconds. It could be that the reader detected a signal but that data point did not transmit through Bluetooth correctly. Results are shown in the tables below. Table 1 shows analytical sensitivity.

TABLE 1
Analytical Sensitivity

| Stock 1 | | | | | Stock 2 | | | | |
|---|---|---|---|---|---|---|---|---|---|
| Concentration | Time | Control | Test | Remarks | Concentration | Time | Control | Test | Remarks |
| 1E5 org per swab | 60 | 8635 | 2056 | n = 1 | 5E3 org per | 231 | 41548 | 1403 | n = 1 |
| | | | | | swab | | | | |
| 1E5 org per swab | 62 | 7706 | 2055 | n = 2 | 5E3 org per | 201 | 39064 | 1096 | n = 2 |
| | | | | | swab | | | | |
| 1E5 org per swab | 61 | 7706 | 2350 | n = 3 | 5E3 org per | 236 | 45129 | 1109 | n = 3 |
| | | | | | swab | | | | |
| 5E4 org per swab | 101 | 8781 | 2085 | n = 1 | 1E3 org per | 346 | 49333 | 1019 | n = 1 |
| | | | | | swab | | | | |
| 5E4 org per swab | 80 | 16335 | 2065 | n = 2 | 1E3 org per | 326 | 47138 | 0 | n = 2 |
| | | | | | swab | | | | |
| 5E4 org per swab | 70 | 8529 | 1682 | n = 3 | 1E3 org per | 297 | 48149 | 1037 | n = 3 |
| | | | | | swab | | | | |
| 1E4 org per swab | 85 | 15341 | 1557 | n = 1 | 500 org per | 360 | 50570 | 0 | n = 1 |
| | | | | | swab | | | | |
| 1E4 org per swab | 106 | 22229 | 1655 | n = 2 | 500 org per | 363 | 49915 | 0 | n = 2 |
| | | | | | swab | | | | |
| 1E4 org per swab | 90 | 19117 | 1876 | n = 3 | | | | | |
| 5E3 org per swab | 135 | unknown | 1428 | n = 1 | | | | | |
| 5E3 org per swab | 103 | 20777 | 1355 | n = 2 | | | | | |
| 5E3 org per swab | 101 | unknown | 1434 | n = 3 | | | | | |
| 1E3 org per swab | 180 | 37764 | 1160 | n = 1 | | | | | |
| 1E3 org per swab | 186 | 31612 | 1809 | n = 2 | | | | | |
| 1E3 org per swab | 148 | 33249 | 1297 | n = 3 | | | | | |
| 500 org per swab | 221 | unknown | 1253 | n = 1 | | | | | |
| 500 org per swab | 185 | 32324 | 1220 | n = 2 | | | | | |
| 500 org per swab | 220 | 42835 | 1363 | n = 3 | | | | | |
| 100 org per swab | 348 | unknown | 1229 | n = 1 | | | | | |
| 100 org per swab | 357 | 49712 | 1190 | n = 2 | | | | | |
| negative | 363 | 45312 | 0 | n = 1 | | | | | |
| negative | 363 | 43331 | 0 | n = 2 | | | | | |
| negative | 363 | 44930 | 0 | n = 3 | | | | | |

### Example 3

### Specificity Study

Twenty presumed negative in-house throat swabs were evaluated on the instrument based strep A test according to the above method. The assay was stopped (if there was no signal above 1000-1200 units) at 6 minutes total time (340 seconds+20 seconds incubation time). The results of the twenty in-house presumed negative throat swabs are recorded in table 2. None of the throat swabs generated a signal greater than 1000-1200 units during the analysis. The instrument-based Strep A test was determined to have no specificity problems with the in-house presumed negative throat swabs.

TABLE 2
Specificity results for 20 Presumed Negative Throat Swabs

| | Time Test | Time Stopped + 20 s | | |
|---|---|---|---|---|
| Sample | Stopped (s) | incubation | Signal | Result |
| 539431 | 340 | 360 | 0 | Negative |
| 625566 | 340 | 360 | 0 | Negative |
| 609281 | 340 | 360 | 0 | Negative |
| 404048 | 340 | 360 | 0 | Negative |
| 500021 | 340 | 360 | 0 | Negative |
| 913449 | 340 | 360 | 0 | Negative |
| 359936 | 340 | 360 | 0 | Negative |
| 400498 | 340 | 360 | 0 | Negative |
| 333219 | 340 | 360 | 0 | Negative |
| 800111 | 340 | 360 | 0 | Negative |
| 498060 | 340 | 360 | 0 | Negative |
| 220986 | 340 | 360 | 0 | Negative |
| 252785 | 340 | 360 | 0 | Negative |
| 421321 | 340 | 360 | 0 | Negative |
| 544442 | 340 | 360 | 0 | Negative |
| 618880 | 340 | 360 | 0 | Negative |
| 204955 | 340 | 360 | 0 | Negative |
| 447888 | 340 | 360 | 0 | Negative |
| 263462 | 340 | 360 | 0 | Negative |
| 660880 | 340 | 360 | 0 | Negative |

### Example 4

### Clinical Study

A collection of twenty-six positive and sixty-five negative clinical samples obtained during the 2007-2008 season were stored at −80° C. and evaluated on the instrument-based Strep A test. All swabs were streaked onto culture plates before being frozen. Table 3 compares results against culture and against the BinaxNOW® Strep A Test. The assay was stopped (if there was no signal above 1000-1200 units) at 6 minutes total time (340 seconds+20 seconds incubation time). Results at different time points are recorded. Table 4 shows sensitivity and specificity at different time points. Table 5 shows sensitivity and specificity with PCR referee. The instrument-based Strep A test was successful in detecting Strep A in clinical samples. It appears to be so sensitive that it even detects real infections that culture misses. When compared against the BinaxNOW® test, it is clear to see that this test is a major improvement from the current test.

TABLE 3
Results against Culture

| | | | | Seconds | | |
|---|---|---|---|---|---|---|
| | | | | after | | |
| | Sam- | | Reader | 20 sec | | BNX |
| Site | ple | TS Cul | Result | incubation | TS cul | −/+ |
| 1 | 41 | Negative | Correct | 340 | 0 | N |
| 2 | 64 | Negative | Correct Until | 309 | 0 | N |
| 2 | 65 | Negative | Correct | 340 | 0 | N |
| 2 | 66 | Negative | Correct | 340 | 0 | N |
| 2 | 67 | Negative | Correct Until | 169 | 0 | N |
| 2 | 68 | Negative | Correct | 340 | 0 | N |
| 2 | 69 | Positive | Correct | 78 | 4+ | P |
| 2 | 70 | Negative | Correct | 340 | 0 | N |
| 2 | 71 | Negative | Correct | 340 | 0 | N |
| 2 | 72 | Negative | Correct | 340 | 0 | N |
| 2 | 73 | Negative | Correct | 340 | 0 | N |
| 2 | 75 | Negative | Correct | 340 | 0 | N |
| 2 | 77 | Negative | Correct | 340 | 0 | N |
| 2 | 78 | Negative | Correct | 340 | 0 | N |
| 2 | 79 | Negative | Correct | 340 | 0 | N |
| 2 | 80 | Negative | Correct | 340 | 0 | N |
| 2 | 81 | Negative | Correct Until | 316 | 0 | N |
| 2 | 82 | Negative | Correct | 340 | 0 | N |
| 2 | 84 | Negative | Correct | 340 | 0 | N |
| 2 | 85 | Negative | Correct | 340 | 0 | N |
| 2 | 86 | Negative | Correct | 340 | 0 | N |
| 2 | 87 | Positive | Correct | 39 | 2+ | N |
| 2 | 88 | Negative | Correct | 340 | 0 | N |
| 2 | 89 | Negative | Correct | 340 | 0 | N |
| 2 | 90 | Negative | Correct | 340 | 0 | N |
| 4 | 111 | Positive | Incorrect | 340 | 2+ | N |
| 4 | 113 | Negative | Incorrect | 43 | 0 | P |
| 4 | 114 | Positive | Correct | 64 | 4+ | P |
| 4 | 115 | Negative | Correct | 340 | 0 | N |
| 4 | 116 | Negative | Correct Until | 145 | 0 | P |
| 4 | 117 | Negative | Correct Until | 215 | 0 | N |
| 4 | 118 | Negative | Correct | 340 | 0 | N |
| 4 | 119 | Negative | Correct | 340 | 0 | N |
| 4 | 120 | Negative | Correct | 340 | 0 | N |
| 4 | 121 | Negative | Correct | 340 | 0 | N |
| 4 | 123 | Positive | Correct | 74 | 4+ | P |
| 4 | 136 | Negative | Correct | 340 | 0 | N |
| 4 | 145 | Positive | Correct | 38 | 4+ | N |
| 4 | 152 | Positive | Correct | 37 | 4+ | P |
| 4 | 163 | Positive | Correct | 38 | 3+ | P |
| 6 | 52 | Negative | Correct | 340 | 0 | N |
| 6 | 53 | Positive | Correct | 99 | 3+ | N |
| 6 | 56 | Positive | Correct | 37 | 3+ | N |
| 6 | 57 | Positive | Correct | 40 | 4+ | P |
| 6 | 58 | Negative | Correct | 340 | 0 | N |
| 6 | 59 | Negative | Correct | 340 | 0 | N |
| 6 | 60 | Negative | Correct | 340 | 0 | N |
| 6 | 61 | Negative | Correct | 340 | 0 | N |
| 6 | 64 | Negative | Correct | 340 | 0 | N |
| 6 | 67 | Negative | Correct | 340 | 0 | N |
| 6 | 68 | Negative | Correct | 340 | 0 | N |
| 6 | 72 | Positive | Correct | 74 | 4+ | P |
| 6 | 73 | Positive | Correct | 40 | 4+ | P |
| 6 | 87 | Positive | Correct | 40 | 4+ | P |
| 6 | 91 | Positive | Correct | 40 | 4+ | P |
| 6 | 94 | Positive | Correct | 141 | 2+ | N |
| 6 | 110 | Negative | Correct | 340 | 0 | N |
| 6 | 113 | Negative | Correct | 340 | 0 | N |
| 6 | 120 | Negative | Correct | 340 | 0 | N |
| 6 | 121 | Positive | Incorrect | 340 | 2+ | N |
| | | | | | (none in 1) | |
| 6 | 122 | Positive | Correct | 249 | 2+ | N |
| | | | | | (none in 1) | |
| 6 | 125 | Negative | Correct Until | 116 | 0 | N |
| 6 | 126 | Negative | Correct | 340 | 0 | N |
| 6 | 127 | Negative | Correct | 340 | 0 | N |
| 6 | 128 | Positive | Correct | 47 | 3+ | P |
| 6 | 129 | Negative | Correct | 340 | 0 | N |
| 6 | 130 | Negative | Correct | 340 | 0 | N |
| 6 | 131 | Positive | Correct | 40 | 4+ | N |
| 6 | 132 | Positive | Correct | 61 | 2+ | N |
| 6 | 136 | ? | ? | 40 | NOT | |
| | | | | | Counted | |
| | | | | | below | |
| 6 | 136 | ? | ? | 129 | NOT | |
| | | | | | Counted | |
| | | | | | below | |
| 6 | 137 | Negative | Correct | 340 | 0 | N |
| 6 | 139 | Negative | Correct | 340 | 0 | N |
| 6 | 140 | Negative | Correct | 340 | 0 | N |
| 6 | 144 | Negative | Correct | 340 | 0 | N |
| 6 | 145 | Negative | Correct | 340 | 0 | N |
| 6 | 146 | Negative | Correct | 340 | 0 | N |
| 6 | 147 | Negative | Correct | 340 | 0 | N |
| 6 | 148 | Negative | Correct | 340 | 0 | N |
| 6 | 149 | Negative | Correct | 340 | 0 | N |
| 6 | 151 | Negative | Correct | 340 | 0 | N |
| 6 | 152 | Negative | Correct | 340 | 0 | N |
| 6 | 153 | Negative | Correct | 340 | 0 | N |
| 6 | 154 | Negative | Correct | 340 | 0 | N |
| 6 | 155 | Negative | Correct | 340 | 0 | N |
| 6 | 157 | Negative | Correct | 340 | 0 | N |
| 6 | 165 | Negative | Correct Until | 308 | 0 | N |
| 6 | 170 | Positive | Correct | 83 | 2+ | N |
| | | | | | (? In 1) | |
| 6 | 171 | Positive | Correct | 43 | 4+ | P |
| 6 | 177 | Positive | Correct | 36 | 2+ | N |
| | | | | | (none in 1) | |
| 6 | 184 | Positive | Correct | 64 | 4+ | P |
| 6 | 187 | Negative | Correct | 340 | 0 | N |
| 6 | 196 | Positive | Correct | 38 | 4+ | P |

TABLE 4
Sensitivity/Specificity at different time points
100 sec run time (2 min total time)

| | | |
|---|---|---|
| Sensitivity | 85% | Missed 4 at 100 sec |
| Specificity | 98% | Missed 1 at 100 sec |

160 sec run time (3 min total time)

| | | |
|---|---|---|
| Sensitivity | 88% | Missed 3, at 160 sec |
| Specificity | 94% | Missed 4, at 160 sec |

280 sec run time (5 min total time)

| | | |
|---|---|---|
| Sensitivity | 92% | Missed 2, at 280 sec |
| Specificity | 92% | Missed 5, at 280 sec |

TABLE 5
Sensitivity/Specificity with PCR Referee

| | | | | BNX NOW | | |
|---|---|---|---|---|---|---|
| Site | ID | Saliva Culture | TS culture | result | BDR result | PCR result |
| 2 | 64 | N | N | N | P at 309 s | INHIBITED |
| 2 | 67 | N | N | N | P at 169 s | INHIBITED |
| 2 | 81 | N | N | N | P at 316 s | INHIBITED |
| 4 | 111 | N | 2+ | N | N | INHIBITED |
| 4 | 113 | N | N | P | P at 43 s | P |
| 4 | 115 | 3+ | N | N | N | N |
| 4 | 116 | N | N | P | P 145 s | P |
| 4 | 117 | N | N | N | P 215 s | P |
| 4 | 121 | 3+ | N | N | N | INHIBITED |
| 4 | 136 | 2+ | N | N | N | N |
| 6 | 121 | 1+ | 2+ (none in | N | N | INHIBITED |
| | | | 1) | | | |
| 6 | 122 | N | 2+, None in | N | P at 249 s | P |
| | | | 1 | | | |
| 6 | 125 | N | N | N | P at 116 | P |
| 6 | 165 | N | N | N | P at 308 | INHIBITED |

at time = 100 seconds (or the read that was closest
to 100 s without going under 100 s)

| | | |
|---|---|---|
| Sensitivity = | 76.70% | 23 out of 30 |
| Specificity = | 100% | 61 out of 61 |

at time = 160 seconds (or the read that was closest
to 160 s without going under 160 s)

| | |
|---|---|
| Sensitivity = | 26 out of 30 |
| 86.6% | |
| Specificity = | 60 out of 61 |
| 98.3% | |

at time = 280 s (or the read closest to 280 s without going under 280 s)

| | |
|---|---|
| Sensitivity = | 28 out of 30 |
| 93.3% | |
| Specificity = | 60 out of 61 |
| 98.3% | |

### Example 5

### Comparison of Low-Cost Instrument to Gold Standard Instrument

The dye Congo red was diluted and sprayed onto nitrocellulose using a Biodot XYZ. The RTG (SN2) was compared against the NES Unipath QC reader. Machine % CV (reading the same strip n=20 times) was compared at a variety of dilutions. Strip to strip % CV was compared at a variety of dilutions. Table 6 shows the machine % CV of SN2 (RTG). Table 7 shows the machine % CV of NES Unipath QC Reader. Table 8 shows the strip to strip % CV at high dose (SN2 Vs NES). Table 9 shows the strip to strip % CV at mid-low dose (SN2 Vs NES). Table 10 shows the strip to strip % CV at low-zero dose (SN2 Vs NES). The data showed that the RTG (SN2) is equivalent in sensitivity and precision to the NES Unipath QC Reader. The RTG (SN2) does seem to have less of a linear response but this can be changed and improved later for quantitative tests. It does not have an impact on the Strep A result.

TABLE 6
Machine % CV of SN2 (RTG)

| | Sn2 | | | | | |
|---|---|---|---|---|---|---|
| | 0.1 mg/mL | 0.05 mg/mL | 0.025 mg/mL | 0.01 mg/mL | 0.005 mg/mL | 0 mg/mL |
| | 9963 | 8040 | 4861 | 1485 | 671 | 0 |
| | 9901 | 8133 | 4850 | 1526 | 648 | 0 |
| | 9940 | 8164 | 4834 | 1508 | 706 | 0 |
| | 9931 | 8067 | 4877 | 1489 | 697 | 0 |
| | 9954 | 8134 | 4800 | 1514 | 677 | 0 |
| | 9921 | 7950 | 4857 | 1459 | 687 | 0 |
| | 9954 | 8120 | 4824 | 1461 | 700 | 0 |
| | 10049 | 8116 | 4888 | 1501 | 744 | 0 |
| | 9954 | 8110 | 4870 | 1475 | 676 | 0 |
| | 9921 | 8106 | 4856 | 1517 | 718 | 0 |
| | 9954 | 8150 | 4781 | 1461 | 694 | 0 |
| | 10035 | 8169 | 4830 | 1464 | 715 | 0 |
| | 9988 | 8160 | 4777 | 1482 | 692 | 0 |
| | 9922 | 8087 | 4786 | 1522 | 682 | 0 |
| | 9966 | 8127 | 4818 | 1484 | 697 | 0 |
| | 9938 | 8131 | 4813 | 1486 | 640 | 0 |
| | 9971 | 8093 | 4804 | 1486 | 687 | 0 |
| | 9969 | 7995 | 4799 | 1494 | 764 | 0 |
| | 9977 | 8092 | 4817 | 1456 | 679 | 0 |
| | 9925 | 8152 | 4814 | 1496 | 646 | 0 |
| Avg | 9957 | 8105 | 4828 | 1488 | 691 | 0 |
| STD | 36 | 55 | 32 | 21 | 29 | 0 |
| % CV | 0.36% | 0.68% | 0.66% | 1.42% | 4.27% | NA |

TABLE 7
Machine % CV of NES Unipath QC Reader

| | NES Area | | | | | |
|---|---|---|---|---|---|---|
| | 0.1 mg/mL | 0.05 mg/mL | 0.025 mg/mL | 0.01 mg/mL | 0.005 mg/mL | 0 mg/mL |
| | 1019 | 604 | 284 | 78 | 33 | 0.00 |
| | 1017 | 603 | 288 | 79 | 34 | 0.00 |
| | 1016 | 603 | 289 | 79 | 35 | 0.00 |
| | 1016 | 602 | 285 | 80 | 38 | 0.00 |
| | 1015 | 603 | 286 | 78 | 33 | 0.00 |
| | 1015 | 602 | 285 | 78 | 33 | 0.00 |
| | 1014 | 600 | 293 | 79 | 31 | 0.00 |
| | 1014 | 600 | 284 | 82 | 33 | 0.00 |
| | 1011 | 601 | 285 | 79 | 33 | 0.00 |
| | 1013 | 599 | 284 | 80 | 31 | 0.00 |
| | 1009 | 599 | 286 | 81 | 34 | 0.00 |
| | 1010 | 600 | 286 | 79 | 32 | 0.00 |
| | 1008 | 597 | 288 | 79 | 32 | 0.00 |
| | 1010 | 599 | 294 | 78 | 33 | 0.00 |
| | 1008 | 598 | 285 | 79 | 36 | 0.00 |
| | 1008 | 596 | 286 | 78 | 33 | 0.00 |
| | 1008 | 598 | 292 | 79 | 33 | 0.00 |
| | 1007 | 597 | 290 | 80 | 33 | 0.00 |
| | 1009 | 595 | 293 | 78 | 32 | 0.00 |
| | 1007 | 594 | 294 | 79 | 31 | 0.00 |
| Avg | 1012 | 599 | 288 | 79 | 33 | 0 |
| STD | 4 | 3 | 3 | 1 | 2 | 0 |
| % CV | 0.37% | 0.45% | 1.20% | 1.09% | 4.76% | NA |

TABLE 8
Strip to Strip % CV at high dose (SN2 Vs NES)

| | | NES | | | NES | |
|---|---|---|---|---|---|---|
| | SN2 | PH | Area | SN2 | PH | Area |
| | 0.1 mg/mL | 0.1 mg/mL | | 0.05 mg/mL | 0.05 mg/mL | |
| | 9737 | 20.5 | 1032 | 8044 | 12.65 | 571.27 |
| | 9830 | 20.2 | 1010 | 8211 | 13.20 | 556.76 |
| | 10208 | 20.7 | 1008 | 8087 | 12.64 | 577.95 |
| | 10352 | 20.4 | 995 | 8224 | 12.74 | 564.15 |
| | 9341 | 20.0 | 1051 | 8114 | 13.31 | 611.44 |
| | 9924 | 20.8 | 1034 | 7886 | 12.45 | 566.06 |
| | 10143 | 20.7 | 1026 | 7969 | 13.09 | 565.26 |
| | 10423 | 19.4 | 980 | 7847 | 12.82 | 572.79 |
| | 9277 | 18.9 | 1016 | 8079 | 13.35 | 590.11 |
| | 10627 | 19.8 | 943 | 8102 | 12.89 | 587.67 |
| | 9803 | 19.7 | 1023 | 8047 | 13.30 | 604.59 |
| | 9821 | 19.6 | 1020 | 7876 | 12.97 | 593.78 |
| | 10153 | 21.1 | 1026 | 8136 | 12.89 | 578.77 |
| | 10471 | 20.4 | 971 | 7921 | 12.71 | 565.74 |
| | 10209 | 20.7 | 1013 | 8196 | 13.17 | 593.13 |
| | 9256 | 19.0 | 1050 | 8054 | 12.80 | 585.80 |
| | 9107 | 19.7 | 1024 | 7984 | 12.92 | 587.20 |
| | 10070 | 20.3 | 999 | 7942 | 13.00 | 575.34 |
| | 9811 | 20.5 | 1029 | 7985 | 12.73 | 528.77 |
| | 10427 | 20.6 | 975 | 7906 | 12.84 | 588.38 |
| Avg | 9950 | 20.1 | 1011 | 8031 | 12.9 | 578 |
| STD | 430 | 1 | 27 | 112 | 0 | 18 |
| % CV | 4.3% | 2.9% | 2.6% | 1.4% | 1.9% | 3.1% |

TABLE 9
Strip to Strip % CV at mid-low dose (SN2 Vs NES)

| | | NES | | | NES | |
|---|---|---|---|---|---|---|
| | SN2 | PH | Area | SN2 | PH | Area |
| | 0.025 mg/mL | 0.025 mg/mL | | 0.01 mg/mL | 0.01 mg/mL | |
| | 4754 | 7.88 | 288% | 1526 | 2.31 | 80 |
| | 4898 | 8.14 | 299 | 1462 | 2.67 | 85 |
| | 4946 | 8.01 | 284 | 1565 | 2.64 | 83 |
| | 4936 | 7.99 | 285 | 1465 | 2.64 | 88 |
| | 4827 | 8.08 | 289 | 1473 | 2.69 | 86 |
| | 4724 | 7.82 | 282 | 1652 | 2.60 | 88 |
| | 4814 | 7.80 | 294 | 1535 | 2.58 | 85 |
| | 4776 | 7.94 | 294 | 1484 | 2.41 | 80 |
| | 4826 | 7.79 | 287 | 1512 | 2.59 | 86 |
| | 4908 | 8.42 | 278 | 1550 | 2.61 | 87 |
| | 4789 | 7.83 | 295 | 1483 | 2.48 | 78 |
| | 4786 | 7.92 | 292 | 1528 | 2.62 | 85 |
| | 4726 | 8.03 | 297 | 1544 | 2.88 | 87 |
| | 4954 | 8.00 | 284 | 1548 | 2.64 | 87 |
| | 4769 | 7.80 | 287 | 1395 | 2.54 | 83 |
| | 4766 | 7.80 | 290 | 1495 | 2.52 | 82 |
| | 4803 | 7.78 | 284 | 1486 | 2.61 | 83 |
| | 4866 | 8.13 | 280 | 1498 | 2.61 | 91 |
| | 4801 | 8.10 | 296 | 1505 | 2.64 | 83 |
| | 4891 | 7.76 | 284 | 1463 | 2.53 | 84 |
| Avg | 4828 | 7.9 | 288 | 1508 | 2.59 | 85 |
| STD | 71 | 0 | 6 | 51 | 0 | 3 |
| % CV | 1.5% | 2.1% | 2.0% | 3.4% | 4.3% | 3.8% |

TABLE 10
Strip to Strip % CV at low-Zero dose (SN2 Vs NES)

| | | NES | | | NES | |
|---|---|---|---|---|---|---|
| | SN2 | PH | Area | SN2 | PH | Area |
| | 0.005 mg/mL | 0.005 mg/mL | | 0 mg/mL | 0 mg/mL | |
| | 628 | 1.14 | 30.13 | 0 | 0.00 | 0.00 |
| | 619 | 1.12 | 28.30 | 0 | 0.00 | 0.00 |
| | 595 | 1.46 | 33.71 | 0 | 0.00 | 0.00 |
| | 606 | 1.04 | 31.27 | 0 | 0.00 | 0.00 |
| | 566 | 1.09 | 30.70 | 0 | 0.00 | 0.00 |
| | 708 | 1.11 | 29.09 | 0 | 0.00 | 0.00 |
| | 794 | 1.04 | 27.20 | 0 | 0.00 | 0.00 |
| | 681 | 1.03 | 27.20 | 0 | 0.00 | 0.00 |
| | 639 | 0.99 | 24.26 | 0 | 0.00 | 0.00 |
| | 702 | 1.23 | 34.66 | 0 | 0.00 | 0.00 |
| | 698 | 1.06 | 28.60 | 0 | 0.00 | 0.00 |
| | 685 | 1.18 | 35.62 | 0 | 0.00 | 0.00 |
| | 614 | 1.16 | 31.13 | 0 | 0.00 | 0.00 |
| | 608 | 0.97 | 28.77 | 0 | 0.00 | 0.00 |
| | 603 | 1.14 | 35.15 | 0 | 0.00 | 0.00 |
| | 619 | 1.07 | 27.38 | 0 | 0.00 | 0.00 |
| | 689 | 1.52 | 45.54 | 0 | 0.00 | 0.00 |
| | 653 | 1.06 | 31.55 | 0 | 0.00 | 0.00 |
| | 665 | 1.06 | 30.03 | 0 | 0.00 | 0.00 |
| | 577 | 0.99 | 27.03 | 0 | 0.00 | 0.00 |
| Avg | 647 | 1.12 | 31 | 0 | 0.00 | 0 |
| STD | 54 | 0 | 4 | 0 | 0 | 0 |
| % CV | 8.3% | 12.3% | 14.4% | NA | NA | NA |

We claim:

1. A device, comprising:
a cassette portion, the cassette portion comprising a bottom, a top, and a reaction chamber, the reaction chamber having an applicator receiving portion for receiving a sample applicator;
a test strip located between the bottom and the top of the cassette portion;
a passage connecting the reaction chamber and the test strip; and
a lateral sliding valve laterally moveable from a first position to a second position, wherein in the first position, the laterally sliding valve obstructs the passage, and wherein in the second position, the laterally sliding valve allows sample to flow through the passage from the reaction chamber to the test strip.

2. The device of claim 1, wherein the top of the cassette portion has a test strip window for viewing a portion of the test strip.

3. The device of claim 1, wherein the reaction chamber is cylindrical.

4. The device of claim 1, wherein the reaction chamber has a grooved wall.

5. The device of claim 1, wherein the reaction chamber further comprises a sample mixing apparatus.

6. The device of claim 1, wherein the reaction chamber contains a labeled antibody pellet.

7. The device of claim 6, wherein the sample mixing apparatus comprises a magnet and a magnetic stirring motor designed to mix the labeled antibody pellet and the sample.

8. The device of claim 1, wherein the test strip comprises a sample receiving pad, a nitrocellulose membrane, and an absorbent pad.

9. The device of claim 5, wherein the test strip further comprises a bridge pad.

10. The device of claim 1, wherein the top cassette further comprises at least one 2D barcode label.

11. The device of claim 5, wherein the at least one 2D barcode label is located on a side of the test strip window.

12. The device of claim 1, wherein the applicator receiving portion of the reaction chamber is a swab cone for receiving a swab applicator.

13. The device of claim 1, wherein the applicator receiving portion of the reaction chamber is a saliva net for receiving a saliva collector.

14. The device of claim 1, wherein the applicator receiving portion is a liquid filter.

15. The device of claim 1, wherein the applicator receiving portion is a buffer pad.

16. The device of claim 1, wherein the applicator receiving portion is at least one blood separation pad.

17. The device of claim 1, wherein the linear sliding valve is moved by a stepper motor.

18. A device, comprising:
a cassette portion, the cassette portion comprising a bottom, a top, and a chamber, the chamber having an applicator receiving portion for receiving a sample applicator;
a test strip located between the bottom and the top of the cassette portion;
a passage connecting the chamber and the test strip;
a valve laterally moveable from a first position to a second position, wherein in the first position, the valve obstructs the passage, and wherein in the second position, the valve allows sample to flow through the passage from the reaction chamber to the test strip.

19. A method, comprising:
applying a sample to an applicator receiving portion of a reaction chamber of a test device;
processing the sample in the applicator receiving portion to prepare a processed sample;
mixing the processed sample and a reagent in the reaction chamber to prepare a mixed sample; and
transferring the mixed sample from the reaction chamber to a test strip of the test device by laterally moving a lateral sliding valve in the test device from a first position to a second position.

20. The method of claim 19, wherein processing the sample comprises extracting saliva from a swab or saliva collector.

21. The method of claim 19, wherein processing the sample comprises filtering and buffering a urine sample.

22. The method of claim 19, wherein processing the sample comprises separating red blood cells from a remainder of the sample.

23. The method of claim 19, wherein mixing the processed sample and the reagent further comprises using a magnet located in the reaction chamber and a magnetic stirring motor.

24. The method of claim 19, wherein the lateral sliding valve is moved from the first position to the second position by an instrument.

25. The method of claim 19, further comprising analyzing the test strip using an instrument.

26. The method of claim 25, wherein analyzing the test strip comprises determining a positive result or negative result.

27. The method of claim 25, wherein analyzing the test strip comprises detecting the concentration level of an analyte in the sample.

28. The method of claim 25, wherein analyzing the test strip further comprises reading a barcode.

29. A method, comprising:
applying a sample to an applicator receiving portion of a chamber of a test device;
processing the sample in the applicator receiving portion to prepare a processed sample;
mixing the processed sample and a reagent in the chamber to prepare a mixed sample; and
transferring the mixed sample from the chamber to a test strip of the test device by laterally moving a lateral sliding valve in the test device from a first position to a second position.
